(19) United States
(12) Patent Application Publication
(10) Pub. No.: US 2004/0248092 A1
(43) Pub. Date: Dec. 9, 2004

(54) Methods of screening for parkinsons's disease

(76) Inventors: Jeffrey M Vance, Chapel Hill, NC (US); Margaret A. Pericak-Vance, Chapel Hill, NC (US); William K. Scott, Durham, NC (US); Jeffrey M. Stajich, Hillsborough, NC (US)

Correspondence Address: MYERS BIGEL SIBLEY & SAJOVEC, PO BOX 37428, RALEIGH, NC, 27627, US

(21) Appl. No.: 10/296,096

(22) PCT Filed: May 24, 2001
(86) PCT No.: PCT/US01/16940

Related U.S. Application Data
(60) Provisional application No. 60/208,102, filed on May 26, 2000.
(60) Provisional application No. 60/238,078, filed on Oct. 4, 2000.
(60) Provisional application No. 60/281,965, filed on Apr. 6, 2001.

(51) Int. Cl. C12Q 1/68

(52) U.S. Cl. 435/6

(57) ABSTRACT

Methods of screening a subject for Parkinson's disease comprise detecting the presence or absence of a functional polymorphism associated with a gene linked to Parkinson's disease. The method may be used diagnostically or prognostically, including in clinical trials for the identification of treatments effective for treating patients carrying particular markers for Parkinson's disease.

### RELATED APPLICATIONS

This application claims the benefit of provisional application Ser. No. 60/208,102, filed May 26, 2000, provisional application Ser. No. 60/238,078, filed Oct. 4, 2000, and provisional application Ser. No. 60/281,965, filed Apr. 6, 2001, the disclosures of all of which are incorporated by reference herein in their entirety.

### STATEMENT OF GOVERNMENT SUPPORT

This invention was made with Government support under Grant Nos. NS39764 and NS26630 from the National Institutes of Health. The Government has certain rights in this invention.

### FIELD OF THE INVENTION

This invention concerns methods of screening for Parkinson's disease, particularly late-onset Parkinson's disease, by the screening of genetic risk factors, methods for screening proposed treatments for Parkinson's disease in clinical trials and the like, and methods of treatment identified by the foregoing.

### BACKGROUND OF THE INVENTION

Parkinson's disease (PD) is a progressive degenerative disease of the central nervous system. The risk of developing Parkinson's disease increases with age, and afflicted individuals are usually adults over 40. Parkinson's disease occurs in all parts of the world, and affects more than one million individuals in the United States alone.

While the primary cause of Parkinson's disease is not known, it is characterized by degeneration of dopaminergic neurons of the substantia nigra. The substantia nigra is a portion of the lower brain, or brain stem, that helps control voluntary movements. The shortage of dopanine in the brain caused by the loss of these neurons is believed to cause the observable disease symptoms.

The symptoms of PD vary from patient to patient. The most common symptom is a paucity of movement: That is, rigidity characterized by an increased stiffness of voluntary skeletal muscles. Additional symptoms include resting tremor, bradykinesia (slowness of movement), poor balance, and walking problems. Common secondary symptoms include depression, sleep disturbance, dizziness, stooped posture, dementia, and problems with speech, breathing, and swallowing. The symptoms become progressively worse and ultimately result in death.

Surgical treatments available for PD include pallidotomy, brain tissue transplants, and deep brain stimulation. Such treatments are obviously highly invasive procedures accompanied by the usual risks of brain surgery, including stroke, partial vision loss, speech and swallowing difficulties, and confusion.

A variety of chemotherapeutic treatments for PD are also available. Perhaps the best known is administration of levodopa, a dopamine precursor. While levodopa administration can result in a dramatic improvement in symptoms, patients can experience serious side-effects, including nausea and vomiting. Concurrent carbidopa administration with levodopa is a significant improvement, with the addition of carbidopa inhibiting levodopa metabolism in the gut, liver and other tissues, thereby allowing more levodopa to reach the brain.

Amantadine hydrochloride is an indirect dopamine agonist (e.g., it either blocks dopamine reuptake or increases dopamine release), and is administered to patients as a monotherapy in the early stages of PD or administered in combination with levodopa (preferably also with carbidopa) as the disease progresses.

Anticholinergic agents such as trihexylphenidyl, benzotropine mesylate, and procyclidine can be administered to PD patients to decrease the activity of cholinergic systems of the brain in a substantially equivalent amount to the decrease experienced by the dopaminergic systems. The restore of a balance of activity between these two competing systems helps alleviate PD symptoms.

Selegiline or deprenyl administration to PD patients delays the need for levodopa administration when prescribed in the earliest stages of PD, and can also be used to boost the effectiveness of levodopa when administered in later stages of the disease.

Dopamine agonists such as bromocriptine, pergolide, pramipexole, and andropinirole are available for treating Parkinson's disease, and can be administered to PD patients either alone or in combination with levodopa.

Catechol-O-methyltransferase (COMT) inhibitors such as tolcapone and entacapone can be administered to PD patients to inhibit COMT, an enzyme which breaks down levodopa before it reaches the brain. Obviously, COMT inhibitors must be used in combination with levodopa administration.

It will be appreciated that PD is unusual among neurodegenerative diseases in that a variety of treatments are available, including treatments that are beneficial in alleviating symptoms at even an early stage of the disease. Accordingly, means for screening subjects for Parkinson's disease would extremely useful in insuring that appropriate treatments are promptly provided.

### SUMMARY OF THE INVENTION

A method of screening a subject for Parkinson's disease is described herein. The method comprises the steps of: detecting the presence or absence of a functional polymorphism associated with a gene linked to Parkinson's disease, with the presence of such a functional polymorphism indicating that the subject is afflicted with or at risk of developing Parkinson's disease. The gene may be the synphilin gene or the ubiquitin conjugating enzyme (UBE2B) gene on chromosome 5, the Parkin gene on chromosome 6, the NAT1 gene or the NAT2 gene on chromosome 8, the proteasome subunits Z or S5 genes or the Torsin A or Torsin B genes on chromosome 9, or the ubiquitin B gene or the Tau gene on chromosome 17. Of course, one, several, or all of the functional polymorphisms associated with all of these genes may be screened in one individual, in one screening session or multiple screening sessions. The detecting step may include detecting whether the subject is heterozygous or homozygous for the functional polymorphism, with subjects who are at least heterozygous for the functional polymorphism being at increased risk for Parkinson's disease.

The step of detecting the presence or absence of the functional polymorphism may include the step of detecting the presence or absence of the functional polymorphism in both chromosomes of the subject (i.e., detecting the presence or absence of one or two alleles containing the functional polymorphism), with two copies of the functional polymorphism (i.e., subjects homozygous for the functional polymorphism) indicating greater risk of Parkinson's disease as compared to heterozygous subjects.

A particular aspect of the present invention is a method of screening a subject for Parkinson's disease comprising detecting the presence or absence of a Parkin gene exon 3 deletion mutation in said subject. The presence of such a deletion mutation indicates that the subject is afflicted with or at risk of developing Parkinson's disease. The deletion mutation typically includes a deletion within base pairs 438-477 (e.g., of at least about 10, 20 or 30 or more bases within this region, optionally overlapping with deletions outside of this region). In one embodiment, the deletion mutation is a deletion of base pairs 438 through 477 inclusive.

A further aspect of the present invention is the use of a means of detecting a mutation as described herein in screening a subject for Parkinson's disease as described herein.

A further aspect of the present invention is a method of screening for susceptibility to Parkinson's Disease in a subject, the method comprising: determining the presence or absence of an allele of a polymorphic marker in the DNA of the patient, wherein (i) the allele is associated with the phenotype of Parkinson's disease, and wherein (ii) the polymorphic marker is within a segment preferably selected from the group consisting of: a segment of chromosome 2 bordered by D2S2982 and D2S1240; a segment of chromosome 2 bordered by D2S1400 and D2S2291; a segment of chromosome 2 bordered by D2S2161 and D2S1334; a segment of chromosome 2 bordered by D2S161 and D2S2297; a segment of chromosome 3 bordered by D3S1554 and D3SS3631; a segment of chromosome 3 bordered by D2S1251 and D3S3546; a segment of chromosome 5 bordered by D5S2064 and D5S1968; a segment of chromosome 5 bordered by D5S2027 and D5S1499; a segment of chromosome 5 bordered by D5S816 and D5S1960; a segment of chromosome 6 bordered by D6S1703 and D6S1027; a segment of chromosome 6 bordered by D6S11581 and D6S2522; a segment of chromosome 8 bordered by D8S504 and D8S258; a segment of chromosome 9 bordered by D9S259 and D9S776; a segment of chromosome 9 bordered by D9S1811 and D9S2168; a segment of chromosome 10 bordered by D10S1122 and D10S1755; a segment of chromosome 11 bordered by D11S4132 and D11S4112; a segment of chromosome 12 bordered by D12S1042 and D12S64; a segment of chromosome 14 bordered by D14S291 and D14S544; a segment of chromosome 17 bordered by D17S1854 and D17S1293; a segment of chromosome 17 bordered by D17S921 and D17S669; a segment of chromosome 21 bordered by D21S1911 and D21S1895; a segment of chromosome 22 bordered by D22S425 and D22S928; a segment of chromosome X bordered by DXS6797 and DXS1205; and a segment of chromosome X bordered by DXS9908 and X telomere; the presence of said allele indicating said subject is at risk of developing Parkinson's disease.

A still further aspect of the present invention is a method of screening a subject for Parkinson's disease, comprising the steps of: detecting the presence or absence of a polymorphism or functional polymorphism associated with a gene linked to Parkinson's disease; the presence of said polymorphism or functional polymorphism indicating said subject is afflicted with or at risk of developing Parkinson's disease; wherein said gene is the tau gene on chromosome 17. In particular examples said polymorphism is IVS3+9A>G (an A to G substitution at a location nine base pairs after the end of intron 3); c1632A>G; c1716T>C; c1761G>A; or IVS11+34G>A.

The foregoing and other objects and aspects of the present invention are explained in detail in the drawings herein and the specification set forth below.

### BRIEF DESCRIPTION OF THE DRAWINGS

FIG. 1 illustrates the Parkin gene exon 3 deletion in a 2% metaphor gel. Controls from the Centre pour l'Etude de Polymorphisme Humain (CEPH) are included to show the results in unaffected individuals without a deletion.

FIG. 2A-C show denaturing high pressure liquid chromatography (dHPLC) tracings from control patients (2A), patients carrying a homozygous Parkin gene exon 3 mutation (2B), and a heterozygous Parkin gene exon 3 mutation (2C).

FIG. 3 also shows dHPLC tracings, but superimposes the tracings from FIG. 2 to allow a direct comparison of the controls, heterozygous deletion, and homozygous deletion dHPLC tracings.

FIG. 4 shows the Parkin gene exon 3 deletion mutation detected in FIGS. 1-3 above. The upper strand shows exon 3 with the deletion present (SEQ ID NO:1), as found in individuals with Parkinson's disease; the lower strand shows exon 3 without the deletion (consensus sequence from controls) (SEQ ID NO:2).

FIG. 5. Two-point maximum heterogeneity lod scores (MHLOD) for all 174 families included in the genomic screen. Each dot represents the MHLOD at each of the. 344 markers analyzed in the genomic screen: Markers with MHLOD >1.5 are labeled.

FIG. 6. Two-point maximum heterogeneity lod scores (MHLOD) for the 156 late-onset families. Each diamond represents the MHLOD at one of the 344 markers analyzed in the genomic screen. Markers with MHLOD >1.5 are labeled.

FIG. 7. Two-point maximum heterogeneity lod scores (MHLOD) for the 18 early-onset families. Each triangle represents the MHLOD at one of the 344 markers analyzed in the genomic screen. Markers with MHLOD >1.5 are labeled.

FIG. 8 is a schematic illustration of a computer assisted method for utilizing markers for Parkinson's disease in identifying treatments for Parkinson's disease.

### DETAILED DESCRIPTION OF THE PREFERRED EMBODIMENTS

The term “Parkinson's disease” as used herein refers to idiopathic Parkinson's disease, or Parkinson's disease of unexplained origin: That is, Parkinson's disease that does not arise from acute exposure to toxic agents, traumatic head injury, or other external insult to the brain. In one embodiment, the invention is particularly concerned with detecting or screening for late onset Parkinson's disease.

The term “late-onset Parkinson's disease” refers to Parkinson's disease which has a time of onset after the subject reaches about 40 years of age.

“Screening” as used herein refers to a procedure used to evaluate a subject for risk of idiopathic Parkinson's disease. It is not required that the screening procedure be free of false positives or false negatives, as long as the screening procedure is useful and beneficial in determining which of those individuals within a group or population of individuals are at increased risk of idiopathic Parkinson's disease. A screening procedure may be carried out for both prognostic and diagnostic purposes (i.e., prognostic methods and diagnostic methods).

“Prognostic method” refers to a method used to help predict, at least in part, the course of a disease. For example, a screening procedure may be carried out on a subject that has not previously been diagnosed with Parkinson's disease, or does not show substantial disease symptoms, when it is desired to obtain an indication of the future likelihood that the subject will be afflicted with Parkinson's disease. In addition, a prognostic method may be carried out on a subject previously diagnosed with Parkinson's disease when it is desired to gain greater insight into how the disease will progress for that particular subject (e.g., the likelihood that a particular patient will respond favorably to a particular drug treatment, or when it is desired to classify or separate Parkinson's disease patients into distinct and different subpopulations for the purpose of conducting a clinical trial thereon). A prognostic method may also be used to determine whether a person will respond to a particular drug.

“Diagnostic method” as used herein refers to a screening procedure carried out on a subject that has previously been determined to be at risk for a particular neurodegenerative disorder due to the presentation of symptoms or the results of another (typically different) screening test

“Functional polymorphism” as used herein refers to a change in the base pair sequence of a gene that produces a qualitative or quantitative change in the activity of the protein encoded by that gene. (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to Parkinson's disease. The term “functional polymorphism” includes mutations.

A “present” functional polymorphism as used herein (e.g., one that is indicative of or a risk factor for Parkinson's disease) refers to the nucleic acid sequence corresponding to the functional polymorphism that is found less frequently in the general population relative to Parkinson's disease as compared to the alternate nucleic acid sequence or sequences found when such functional polymorphism is said to be “absent”.

“Mutation” as used herein sometimes refers to a functional polymorphism that occurs in less than one percent of the population, and is strongly correlated to the presence of a gene (i.e., the presence of such a mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than would be expected by chance, thereby indicating that the gene or genes within the linked chromosome region contain or are associated with a functional polymorphism that is correlated to the presence of, or risk of, disease. Once linkage is established, association studies (linkage disequilibrium) can be used to narrow the region of interest or to identify the risk conferring gene for Parkinson's disease.

“Associated with” when used to refer to a functional polymorphism and a particular gene means that the functional polymorphism is either within the indicated gene, or in a different, physically adjacent, gene on that chromosome. In general, such a physically adjacent gene is on the same chromosome and within 1 or 2 centimorgans of the named gene (i.e., within about 1 or 2 million base pairs of the named gene).

As noted above, the present invention provides a method of screening (e.g., diagnosing or prognosing) for Parkinson's disease in a subject. Subjects with which the present invention is concerned are primarily human subjects, including male and female subjects of any age or race. The method comprises detecting the presence or absence of a functional polymorphism associated with a gene linked to Parkinson's disease as set forth in Table 1.

TABLE 1
Chromosome regions (genes) linked to Parkinson's disease.

| Chromosome | Genes |
|---|---|
| 5 | Synphilin and the ubiquitin conjugating enzyme (UBE2B) |
| 6 | Parkin |
| 8 | NAT1 and NAT2 |
| 9 | Two proteasome subunits (Z and S5) PSMB7, PSMD5; |
| | Torsin A (DYT1) or Torsin B |
| 17 | Ubiquitin B (UBB) and Tau (MAPT) |

The presence of a functional polymorphism associated with a gene linked to Parkinson's disease indicates that the subject is afflicted with Parkinson's disease or is at risk of developing Parkinson's disease. A subject who is “at increased risk of developing Parkinson's disease” is one who is predisposed to the disease, has genetic susceptibility for the disease or is more likely to develop the disease than subjects in which the detected functional polymorphism is absent While the methods described herein may be employed to screen for any type of idiopathic Parkinson's disease, a primary application is in screening for late-onset Parkinson's disease.

Suitable subjects include those who have not previously been diagnosed as afflicted with Parkinson's disease, those who have previously been determined to be at risk of developing Parkinson's disease, and those who have been initially diagnosed as being afflicted with Parkinson's disease where confirming information is desired. Thus it is contemplated that the methods described herein be used in conjunction with other clinical diagnostic information known or described in the art which are used in evaluation of subjects with Parkinson's disease or suspected to be at risk for developing such disease.

The detecting step may be carried out 3 in accordance with known techniques (see, e.g., U.S. Pat. Nos. 6,027,896 and 5,508,167 to Roses et al.), such as by collecting a biological sample containing DNA from the subject, and then determining the presence or absence of DNA encoding or indicative of the functional polymorphism in the biological sample (e.g., the Parkin gene exon 3 deletion mutation described herein). Any biological sample which contains the DNA of that subject may be employed, including tissue samples and blood samples, with blood cells being a particularly convenient source.

Determining the presence or absence of DNA encoding a particular functional polymorphism may be carried out with an oligonucleotide probe labelled with a suitable detectable group, and/or by means of an amplification reaction such as a polymerase chain reaction or ligase chain reaction (the product of which amplification reaction may then be detected with a labelled oligonucleotide probe or a number of other techniques). Further, the detecting step may include the step of detecting whether the subject is heterozygous or homozygous for the particular functional polymorphism. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference).

Amplification of a selected, or target, nucleic acid sequence may be carried out by any suitable means. See generally D. Kwoh and T. Kwoh, Am. Biotechnol. Lab. 8, 1425 (1990). Examples of suitable amplification techniques include, but are not limited to, polymerase chain reaction, ligase chain reaction, strand displacement amplification (see generally G. Walker et al., Proc. Natl. Acad Sci. USA 89, 392-396 (1992); G. Walker et al., Nucleic Acids Res. 20, 1691-1696 (1992)), transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Qβ replicase system (see P. Lizardi et al., BioTechnology 6, 1197-1202 (1988)), nucleic acid sequence-based amplification (or “NASBA”) (see R Lewis, Genetic Engineering News 12 (9), 1 (1992)), the repair chain reaction (or “RCR”) (see R Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

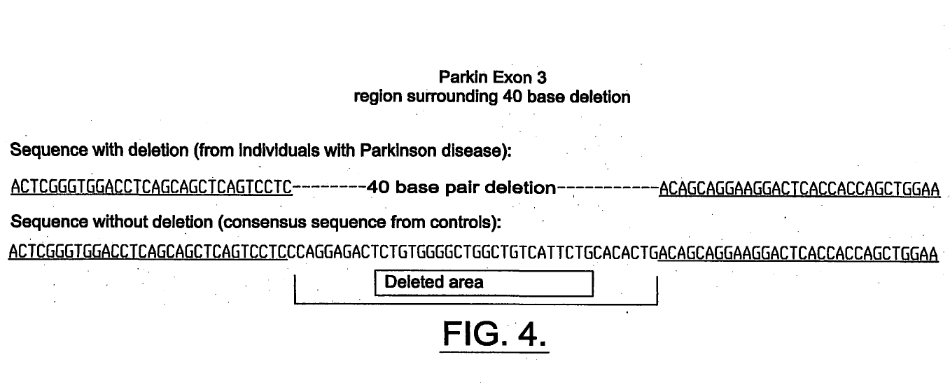

DNA amplification techniques such as, the foregoing can involve the use of a probe or primer, a pair of probes or primers, or two pairs of probes or primers which specifically bind to DNA containing the functional polymorphism, but do not bind to DNA that does not contain the functional polymorphism Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes or primers can be generated in accordance with standard techniques from the known sequences of DNA in or associated with a gene linked to Parkinson's disease (see, e.g., FIG. 4 herein), or from sequences which can be generated from such genes in accordance with standard techniques.

It will be appreciated that the detecting steps described herein may be carried out directly or indirectly. Means of indirectly determining allelic type include measuring polymorphic markers that are linked to the, particular functional polymorphism, as has been demonstrated for the VNTR (variable number tandem repeats) and the ApoB alleles (Decorter et al., DNA & Cell Biology 9(6), 461-69 (1990), and collecting and determining differences in the protein encoded by a gene containing a functional variant, as described for ApoE4 in U.S. Pat. Nos. 5,508,167 and 6,027,896 to Roses et al.

Kits for determining if a subject is or was (in the case of deceased subjects) afflicted with or is or was at increased risk of developing Parkinson's disease will include at least one reagent specific for detecting for the presence or absence of at least one functional polymorphism as described herein and instructions for observing that the subject is or was afflicted with or is or was at increased risk of developing Parkinson's disease if at least one of the functional polymorphisms is detected. The kit may optionally include one or more nucleic acid probes for the amplification and/or detection of the functional polymorphism by any of the techniques described above, with PCR being currently preferred.

Screening by Markers linked to Parkinson 's Disease. The present invention may be carried out by screening for markers within particular segments of DNA as described in (for example) U.S. Pat. No. 5,879,884 to Peroutka (the disclosure of which is incorporated by reference herein in its entirety. Examples of suitable segments are given in Table 2 below.

In general, a method of screening for susceptibility to Parkinson's Disease in a subject comprises determining the presence or absence of an allele of a polymorphic marker in the DNA of the patient, wherein (i) the allele is associated with the phenotype of Parkinson's disease, and wherein (ii) the polymorphic marker is within a segment set forth in column 3 of Table 2 below, or within 5, 10, or 15 centiMorgans (cM) of the markers set forth in column 1 of Table 2 below. The presence of the allele indicates the subject is at risk of developing Parkinson's disease.

To carry out the foregoing, nucleic acid samples can be collected from individuals of a family having multiple individuals afflicted with Parkinson's disease. Linkage within that family is then assessed within the regions set forth above in accordance with known techniques, such as have been employed previously in the diagnosis of disorders such as Huntington's disease, and as described in U.S. Pat. No. No. 5,879,884 to Peroutka. A disadvantage of such procedures is that the degree of confidence in the result may depend upon family size. Accordingly, another way to carry out the foregoing methods is to statistically associate alleles at a marker within the segments described above with Parkinson's disease, and use such alleles in genetic testing in accordance with known procedures, such as described for the polymorphism in Example 5 below in connection with the tau gene.

TABLE 2
Genomic regions generating LOD scores greater
than 1 in the PD genomic screen.

| | 40 cM | | |
|---|---|---|---|
| | Interval on | | |
| | Marshfield | | |
| | 1998 | | |
| | Sex- | Marker | Strata in which |
| | Averaged | boundaries for | interval |
| Peak Marker | Map | 40 cM Interval | has LOD > 1 |
| Chromosome 2 | | | |
| D2S1329 | 0-35 | D2S2982-D2S1240 | Early onset |
| D2S405 | 26-68 | D2S1400-D2S2291 | Early onset |
| D2S410 | 105-145 | D2S2161-D2S1334 | Early onset |
| D2S434 | 192-232 | D2S161-D2S2297 | Dopa responsive* |
| Chromosome 3 | | | |
| D3S1768 | 41-81 | D3S1554-D3S3631 | Non-dopa |
| | | | responsive |
| D3S2460 | 114-154 | D3S1251-D3S3546 | Non-dopa |
| | | | responsive |
| Chromosome 5 | | | |
| D5S2848 | 20-60 | D5S2064-D5S1968 | Overall**, late |
| | | | onset**, Dopa |
| | | | responsive** |
| D5S186 | 119-159 | D5S2027-D5S1499 | Overall, early |
| | | | onset**, late |
| | | | onset**, dopa |
| | | | responsive** |
| D5S1480 | 139-179 | D5S816-D5S1960 | Non-dopa |
| | | | responsive |
| Chromosome 6 | | | |
| D6S305 | 146-186 | D6S1703-D6S1027 | Early onset |
| D6S503 | 164-193 | D6S1581-D6S2522 | Non-dopa |
| | | | responsive |
| Chromosome 8 | | | |
| D8S520 | 0-40 | D8S504-D8S258 | Overall, late- |
| | | | onset, |
| | | | dopa responsive |
| Chromosome 9 | | | |
| D9S301 | 46-86 | D9S259-D9S776 | Non dopa |
| | | | responsive |
| D9S2157 | 126-166 | D9S1811-D9S2168 | Overall, late |
| | | | onset, |
| | | | non-dopa |
| | | | responsive |
| Chromosome 10 | | | |
| D10S1432 | 73-113 | D10S122- | Early onset** |
| | | D10S1755 | |
| Chromosome 11 | | | |
| D11S4131 | 118-147 | D11S4132- | Early onset |
| | | D11S4112 | |
| Chromosome 12 | | | |
| D12S398 | 48-88 | D12S1042-D12S64 | Early onset** |
| Chromosome 14 | | | |
| D1421426 | 105-138 | D14S291-D14S544 | Overall**, late |
| | | | onset**, dopa |
| | | | responsive |
| Chromosome 17 | | | |
| D17S921 | 16-56 | D17S1854- | Overall, early |
| | | D17S1293 | onset |
| D17S1293 | 36-76 | D17S921-D17S669 | Late-onset, |
| | | | dopa responsive |
| Chromosome 21 | | | |
| D21S1437 | 0-33 | D21S1911- | Late onset, |
| | | D21S1895 | dopa responsive |
| Chromosome 22 | | | |
| D22S685 | 12-52 | D22S425-D22S928 | Late onset**, |
| | | | dopa |
| | | | responsive**, |
| | | | non-dopa |
| | | | responsive** |
| Chromosome X | | | |
| GATA165B12 | 113-153# | DXS6796- | Overall**, late- |
| | | DXS1205 | onset**, dopa |
| | | | responsive** |
| DXYS154 | 164-184# | DXS9908-X | Late onset**, |
| | | telomere | dopa |
| | | | responsive** |

*= Multipoint LOD > 1 only
**= Single point LOD > 1 only
#= female map distances

Clinical trials and drug discovery. As noted above, the prognostic methods described herein may also be used to determine whether a person will respond to a particular drug. This is useful, among other things, for matching particular drug treatments to particular patient populations to thereby exclude patients for whom a particular drug treatment may be less efficacious.

Thus the present invention provides a computer assisted method of identifying a proposed treatment for Parkinson's disease (in a human subject). The method involves the steps of (a) storing a database of biological data for a plurality of patients, the biological data that is being stored including for each of said plurality of patients (i) a treatment type, (ii) at least one genetic marker associated with Parkinson's disease, and (iii) at least one disease progression measure for Parkinson's disease from which treatment efficacy may be determined; and then ( ) querying the database to determine the dependence on said genetic marker of the effectiveness of a treatment type in treating Parkinson's disease, to thereby identify a proposed treatment as an effective treatment for a patient carrying a particular marker for Parkinson's disease.

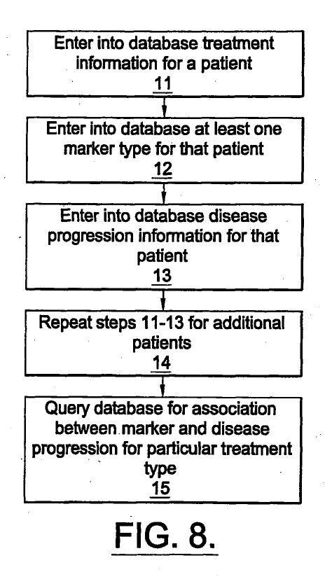

In one embodiment illustrated schematically in FIG. 8, treatment information for a patient is entered into the database 11 (through any suitable means such as a window or text interface), genetic marker information for that patient is entered into the database 12, and disease progression information is entered into the database 13. These steps are then repeated until the desired number of patients have been entered into the database 14. The database can then queried 15 to determine whether a particular treatment is effective for patients carrying a particular marker, not effective for patients carrying a particular marker, etc. Such querying may be carried out prospectively or retrospectively on the database by any suitable means, but is generally done by statistical analysis in accordance with known techniques, as discussed further below.

Any suitable disease progression measure can be used, including but not limited to measures of motor function such as tremor measures, rigidity measures, akinesia measures, and dementia measures, as well as combinations thereof. The measures are preferably scored in accordance with standard techniques for entry into the database. Measures are preferably taken at the initiation of the study, and then during the course of the study (that is, treatment of the group of patients with the experimental and control treatments), and the database preferably incorporates a plurality of these measures taken over time so that the presence, absence, or rate of disease progression in particular individuals or groups of individuals may be assessed.

An advantage of the present invention is the relatively large number of genetic markers for Parkinson's disease (as set forth herein) that may be utilized in the computer-based method. Thus, for example, instead of entering a single marker into the database for each patient, two, three, five, seven or even ten or more markers may be entered for each particular patient Note that, for these purposes, entry of a marker includes entry of the absence of a particular marker for a particular patient. Thus the database can be queried for the effectiveness of a particular treatment in patients carrying any of a variety of markers, or combinations of markers, or who lack particular markers.

In general, the treatment type may be a control treatment or an experimental treatment, and the database preferably includes a plurality of patients having control treatments and a plurality of patients having experimental treatments. With respect to control treatments, the control treatment may be a placebo treatment or treatment with a known treatment for Parkinson's disease, and preferably the database includes both a plurality of patients having control treatment with a placebo and a plurality of patients having control treatments with a known treatment for Parkinson's disease

Experimental treatments are typically drug treatments, which are compounds or active agents that are parenterally administered to the patient (i.e., orally or by injection) in a suitable pharmaceutically acceptable carrier.

Control treatments include placebo treatments (for example, injection with physiological saline solution or administration of whatever carrier vehicle is used to administer the experimental treatment, but without the active agent), as well as treatments with known agents for the treatment of Parkinson's disease, such as administration of Levodopa, amantadine, anticholinergic agents, antihistamines, phenothiazines, centrally acting muscle relaxants, etc. See, e.g., L. Goodman and A. Gilman, The Pharmacological Basis of Therapeutics, 227-244 (5^{th} Ed. 1975).

Administration of the treatments is preferably carried out in a manner so that the subject does not know whether that subject is receiving an experimental or control treatment. In addition, administration is preferably carried out in a manner so that the individual or people administering the treatment to the subject do not know whether that subject is receiving an experimental or control treatment.

Computer systems used to carry out the present invention may be implemented as hardware, software, or both hardware and software. Computer and hardware and software systems that may be used to implement the methods described herein are known and available to those skilled in the art See, e.g., U.S. Pat. No. 6,108,635 to Herren et al. and the following references cited therein: Eas, M. A.: A program for the meta-analysis of clinical trials, Computer Methods and Programs in Biomedicine, vol 53, no. 3 (July 1997); D. Klinger and N Jaffe, An Information Technology Architecturefor Pharmaceutical Research and Development, 14^{th} Annual Symposium on Computer Applications in Medical Care, Nov. 4-7, pp. 256-260 (Washington, D.C. 1990); M. Rosenberg, “ClinAccess: An integrated client/server approach to clinical data management and regulatory approval”, Proceedings of the 21^{st} annual SAS Users Group International Conference (Cary, N.C., March 10-13 1996). Querying of the database may be carried out in accordance with known techniques such as regression analysis or other types of comparisons such as with simple normal or t-tests, or with non-parametric techniques.

The present invention accordingly provides for a method of treating a subject for Parkinson's disease, particularly late-onset Parkinson's disease, which method comprises the steps of: determining the presence of a preselected marker for Parkinson's disease in said subject; and then administering to said subject a treatment effective for treating Parkinson's disease in a subject that carries said marker. The preselected marker is a marker such as described above, but to which a particular treatment has been matched. A treatment is preferably identified for that marker by the computer-assisted method described above. In one a particularly preferred embodiment, the method is utilized to identify patient populations, as delineated by preselected ones of markers such as described herein, for which a treatment is effective, but where that treatment is not effective or is less effective in the general population of Parkinson's disease patient (that is, patients carrying other markers, but not the preselected marker for which the particular treatment has been identified as effective).

The present invention is explained in greater detail in the following non-limiting Examples.

### EXAMPLE 1

### Identification of a Parkin Gene Exon 3 Deletion Mutation in Parkinson's Disease Families

Multiplex sibship familes were collected and a complete genomic screen (N=325 markers; 10 cM grid) was conducted to identify susceptibility genes for familial Parkinson's disease (PD).

Individuals with PD (N=379; mean age of onset (AOO)=60.1±12.7 years) and their families (N=175 families with ≧2 members with PD) were collected from 13 sites using strict consensus clinical criteria This PD dataset is clinically similar to other clinic based populations of Parkinson disease (Hubble et al., Neurology 52, A13 (1999). Several areas of interest were found including the region containing the Parkin gene. Areas that are currently of greatest interest are set forth in Table 1 above.

Subsequent genetic analysis of this data demonstrated a significant genetic effect in individuals with PD in the chromosome 6 region around the Parkin gene. This effect was strongest in families with at least one member with Parkinson disease onset prior to age 40. Age of onset in this subset (N=89) ranged from 12 to 80 years. This subset was then prioritized for screening of the Parkin gene using denaturing high pressure liquid chromatography (dHPLC). Unique changes in 46 of the 88 individuals screened were identified. Analysis of PCR products of exon 3 of one of the changes revealed a small deletion of bases 438 to 477, present in a homozygous and heterozygous state in at least 5 different families (range of AOO: 19-53). Examination of these familes show that they have the same 40 bp deletion for exon 3. They were collected from all over the United States of America Thus this deletion is a relatively common allele in the population, and clearly contributes to PD in the USA, in families not known to have an autosomal recessive inheritance pattern. In fact, the heterozygotes are compound heterozygotes, with a mutation in the other allele in another exon.

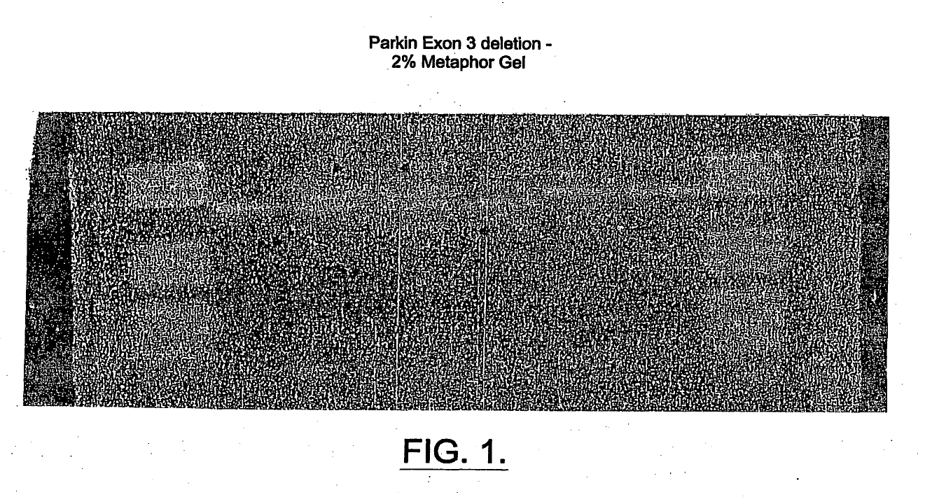

FIG. 1 illustrates the Parkin gene exon 3 deletion in a 2% metaphor gel. This figure illustrates the difference in the size of the PCR products of Parkin exon 3 with and without the 40 base-pair deletion. Deletions in both copies of the Parkin gene (homozygous deletions) result in a single band that travels farther in the gel due to its smaller size. Deletion in only one of the copies (heterozygous deletion) results in two bands —the band that travels farther is the deletion, and the other band is the copy of the gene without the deletion. Controls from the Centre pour l'Etude de Polymorphisme Humain (CEPH) are included to show the results in unaffected individuals without a deletion.

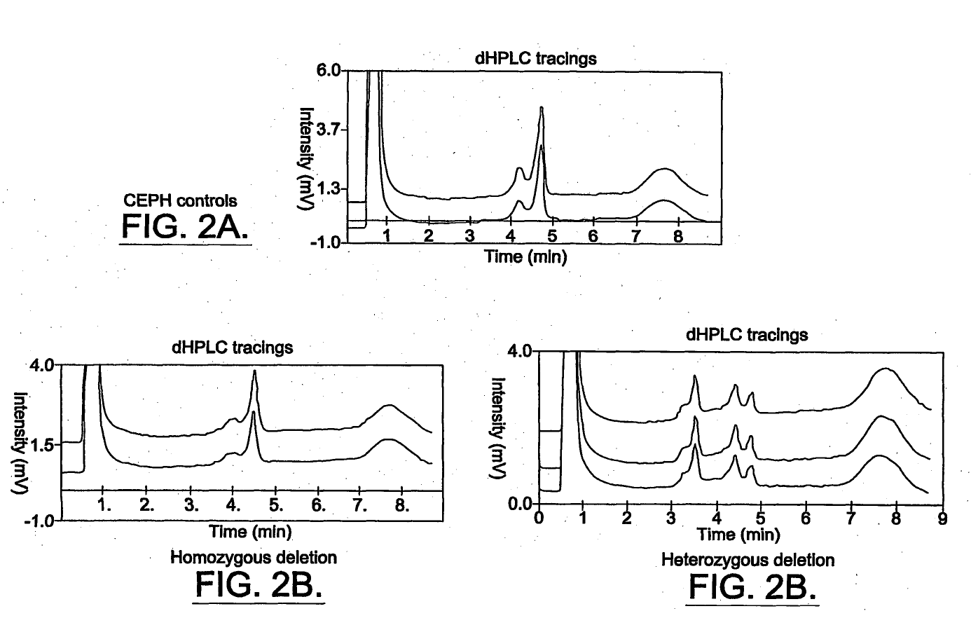

FIG. 2A-C show denaturing high pressure liquid chromatography (dHPLC) tracings from control patients (2A), patients carrying a homozygous Parkin gene exon 3 mutation (2B), and a heterozygous Parkin gene exon 3 mutation (2C). PCR products for exon 3 of Parkin were separated on a dHPLC column and elution times compared among affected individuals and unaffected controls. Differences in the resulting tracings indicate sequence changes that affect the structure of the PCR product and the elution time. The top tracing illustrates the pattern obtained from the CEPH controls (with no deletion). The bottom two tracings illustrate the markedly different patterns obtained from individuals homozygous and heterozygous for the deletion.

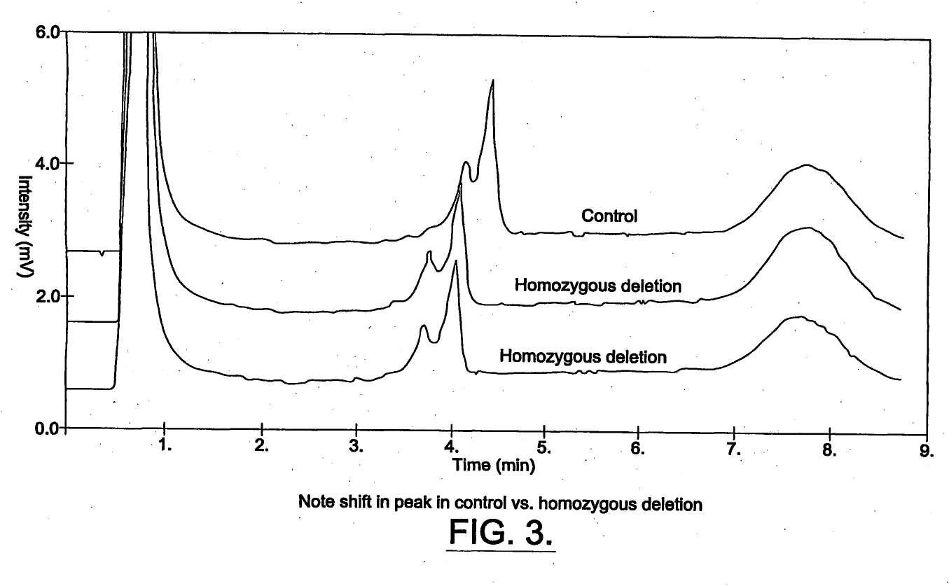

FIG. 3 also shows dHPLC tracings, but superimposes the tracings from FIG. 2 to allow a direct comparison of the controls, heterozygous deletion, and homozygous deletion dHPLC tracings.

FIG. 4 shows the Parkin gene exon 3 deletion mutation detected in FIGS. 1-3 above. The upper strand shows exon 3 with the deletion present, as found in individuals with Parkinson's disease; the lower strand shows exon 3 without the deletion (consensus sequence from controls). Information such as set forth in FIG. 4 may be used to develop oligonucleotide probes useful for detecting functional polymorphisms in screening procedures for particular functional polymorphisms, as set forth in the following Example.

### EXAMPLE 2

### PCR Screening Procedures

Blood or other biological samples containing DNA are obtained from a subject. DNA is extracted from these samples using conventional techniques. Polymerase chain reaction is performed on the genomic DNA of the subject using the primers for Parkin Exon 3 described in T Kitada et al., Nature 392, 605 (1988) (the disclosure of which is incorporated herein by reference), as follows:

| | |
|---|---|
| forward (5′-3′) | |
| ACATGTCACTTTTGCTTCCCT | (SEQ ID NO:3) |
| reverse (5′-3′) | |
| AGGCCATGCTCCATGCAGACTGC | (SEQ ID NO:4) |

The shortened PCR product produced by the 40 base pair exon 3 deletion mutation (bp438-477) (numbering based upon the cDNA of T. Kitada et al., supra) can be detected from the amplification products of such primers by a variety of techniques. For example, agarose gel separation of the PCR products in which two bands would be obtained, the smaller molecular weight band being the one containing the deletion Size of the deletion can be measured using a molecular weight standard. In the alternative, denaturing high performance liquid chromatography (DHPLC) can be used, in which a distinct peak representing the deletion is detected that comes off the column earlier than control peaks. Identification of this specific deletion would require subsequent sequencing of the PCR product

### EXAMPLE 3

### Parkin Mutations and Idiopathic Parkinson Disease

The marker D6SO₃, parkin intron 7, was found in further screening of 174 linked early onset (n=18) and late onset (n=156) Parkinson disease family to be strongly to Parkinson disease, with a peak Lod score of 5.0.

Familial and sporadic PD cases were screened for par in mutations, unselected for age at onset or inheritance pattern. Samples included samples from 88 affected individuals (mean age of onset: 38.6±14.2; selected from 57 families containing individuals with age of onset less than 40; 83% with a reported family history of PD) as well as pools of affected individuals from 308 families (mean age of onset 54.4±13 years; selected individual with earliest age of onset from each family; pools of 5 samples; 97% with reported family history of PD).

A two stage mutation screening strategy was employed, with exons amplified using PCR primers from Hattori et al., Ann. Neurol. 44, 93541 (1998). Products were initially screened using denaturing high-pressure liquid chromatography (HPLC), and DHPLC abnormalities were studied further by sequencing. Results are summarized in Table 3 below (numbering is again based upon the cDNA of T. Kitada et al., supra).

Note that ten distinct mutations were detected, only three of which were previously reported. Two mutations (exon 7, Asp>Asn and exon 3, Ala>Glu) were detected only in late-onset families.

TABLE 3
Parkin mutations detected.

| | Amino Acid | # | # | Mean | | |
|---|---|---|---|---|---|---|
| Nucleotide Change | Change | individuals | families | AO | Range | Ref. |
| Homozygous | Stop | 5 | 2 | 38.0 | 19-53 | |
| 438-477 del 40 bp | | | | | | |
| 438-477 del 40 bp + 1390 | Stop + Gly430Asp | 2 | 1 | 25.5 | 22-29 | Gly > Asp¹ |
| G > A | | | | | | |
| 438-477 del 40 bp | Stop | 9 | 4 | 35.0 | 21-57 | |
| only | | | | | | |
| All 438-477 del 40 bp | Stop | 16 | 7 | 34.8 | 19-57 | |
| 924 C > T + 1412 | Arg275Trp + Pro > Leu | 2 | 1 | 45.0 | 38-52 | Arg > Trp² |
| C > T | | | | | | |
| 924 C > T + 859 | Arg275Trp + Cys > Tyr + Pro > Leu | 2 | 1 | 24.0 | 21-27 | |
| G > A + 1412 | | | | | | |
| C > T | | | | | | |
| 924 C > T only | Arg275Trp | 4 | 4 | 54.0 | 39-71 | |
| | only | | | | | |
| All 924 C > T | All | 8 | 6 | 44.3 | 21-71 | |
| | Arg275Trp | | | | | |
| Homozygous | Gln34/Stop37 | 2 | 1 | 25.5 | 19-32 | Del |
| 202-203 del AG | | | | | | AG² |
| 199 G > A + G > T | Arg > Gln + G > T | 2 | 1 | 16.5 | 12-21 | |
| exon 9 + 4³ | in intron | | | | | |
| 346 C > A | Ala > Glu | 1 | 1 | 62.0 | 62 | |
| 885 G > A | Asp > Asn | 1 | 1 | 52.0 | 52 | |
| All Mutations | | 28 | 17 | 39.6 | 12-71 | |

¹Lucking et al., New England Journal of Medicine 342: 1560-7 (2000)
²Abbas et al., Human Molecular Genetics 8: 567-74 (1999)
³Refers to the position 4 base pairs pat the end of exon 9, e.g., in the intron.

The mutations noted in Table 3 above may be used to carry out the methods described herein.

### EXAMPLE 4

### Genomic Screening for Additional Parkinson's Disease Markers

To identify additional regions of the genome with genes contributing to idiopathic PD, we performed a complete genomic screen for linkage analysis in 174 PD families containing at least one affected relative pair.

A. Methods

Family Ascertainment The Duke Center for Human. Genetics (DCHG)/GlaxoSmithKline/Deane Laboratory Parkinson Disease Genetics Collaboration is a 13-center effort established to ascertain multiplex (2 or more participating individuals diagnosed with PD) families for genetic studies of PD.

Family history of PD was documented for each family by conducting a standard interview with the proband or a knowledgeable family informant. The results of this interview were used to generate pedigrees documenting the extent of family history of PD out to three degrees of relationship (1^{st} cousins). Consensus diagnostic and exclusion criteria were developed by all participating clinicians prior to beginning ascertainment of families. All participants are examined prior to enrollment in the study by a board-certified neurologist or a physician-assistant trained in neurological disease and supervised by a neurologist. Participants are classified as affected, unclear, or unaffected based on-neurological exam and clinical history. Affected individuals possess at least two cardinal signs of PD (rest tremor, bradykinesia, and rigidity) and have no atypical clinical features or other causes of parkinsonism. Unclear individuals possess only one sign and/or have a history of atypical clinical features, and unaffected individuals have no signs of PD. Excluded from participation are individuals with a history of encephalitis, neuroleptic therapy within the year prior to diagnosis, evidence of normal pressure hydrocephalus, or a clinical course with unusual features, suggestive of atypical or secondary parkinsonism. Age at onset was self-reported, defined as the age at which the affected individual could first recall noticing one of the primary signs, of PD. Physician and patient observations of response to levodopa therapy were used to classify individuals as responsive or non-responsive to levodopa. Individuals for whom levodopa was of uncertain benefit or who never received levodopa therapy were classified as having unknown levodopa response. To ensure diagnostic consistency across sites, clinical data for all participants was reviewed by a clinical adjudication board, consisting of a board certified neurologist with fellowship training in movement disorders, a dually board-certified neurologist and Ph.D. medical geneticist, and a certified physician assistant. All participants gave informed consent prior to venipuncture and data collection according to protocols approved by each center's institutional review board.

The first 174 families with sampled affected relative pairs were included in this initial genomic screen. The number of sampled affected family members and affected relative pairs is presented in Table 4. The families contained an average of 2.3 affected individuals and an average of 1.5 affected relative pairs per family. While the majority of the affected relative pairs were affected sibpairs (185/260), there were 75 other affected relative pairs (avuncular, cousin, and parent-child pairs) in the data set. These data illustrate that the while smaller family aggregates without a recognizable mode of inheritance were studied, families were often multigenerational in structure and that the study was not limited to affected sibpairs.

All families studied were Caucasian. Overall, 870 individuals (an average of 5 per family) from these families were studied: 378 affected with PD (43%), 379 unaffected (44%), and 113 with unclear affection status (13%). In affected individuals, the mean age at onset of PD was 59.9±12.6 years (range: 12-90), and the mean age at examination was 69.9±10.2 years (range: 33-90). Mean age of examination in unaffected individuals was 67.1±12.9 years (range 31-96), and mean age of examination in those with unclear affection status was 72.1±11.6 years (range 49-90).

Molecular Analysis. Genomic DNA was extracted from whole blood using Puregene© in methods previously described (J. M. Vance, in Approaches to Gene Mapping in Complex Human Diseases, J. L. Haines and M. A. Pericak-Vance, Eds. (Wiley-Liss, New York, 1998), chap. 8). Analysis was performed on 344 microsatellite markers with an average spacing of 1.0 cM. Genotyping was performed by the FAAST method previously described (J. M. Vance, K. Ben Othmane, in Approaches to Gene Mapping in Complex Human Diseases, J. L. Haines and M. X Pericak-Vance, Eds. (Wiley-Liss, New York, 1998), chap. 9). Systematic genotyping errors were minimized using a system of quality control checks with duplicated samples (J. B. Rimmler et al., Am. J. Hum. Genet. 65 (Supplement), A442 (1999); On each 96-well PCR plate, two standard samples from CEPH families are included and 6 additional samples are duplicates of samples either on that plate or another plate in the screen. Laboratory technicians are blinded to the location of these QC samples to avoid bias in interpretation of results. Automated computer scripts check each set of genotypes submitted by the technician for mis-matches between the duplicated samples; mismatches are indicative of potential genotype reading errors, mis-loading of samples, and sample mix-ups.).

As an additional quality control measure, potential pedigree errors were checked using the program RELPAIR (M. Boebnke, N. J. Cox, Am. J. Hum. Genet. 61, 423 (1997)), which infers likely relationships between pairs of relatives using IBD sharing estimates from a set of microsatellite markers.

Statistical Analysis. Data analysis consisted of a multianalytical approach consisting of both parametric lod score and non-parametric affected relative pair methods. Maximized parametric lod scores (MLOD) for each marker were calculated using the VITESSE and HOMOG program packages (J. R O'Connell, D. E. Weeks, Nat. Genet. 11, 402 (1995); J. Ott, Analysis of Human Genetic Linkage. (The Johns Hopkins University Press, Baltimore, ed. 3, 1999); The MLOD is the lod score maximized over the two genetic models tested, allowing for genetic heterogeneity. Dominant and recessive low-penetrance (affecteds-only) models were considered. Prevalence estimates for PD range from 0.3% in individuals aged 40 and older to 2.5% in individuals aged 70 and older [C. M. Tanner, S. M. Goldman, Neurol. Clin. 14, 317 (1996)]. Based on these prevalence estimates and allowing for age-dependent or incomplete penetrance, disease allele frequencies of 0.001 for the dominant model and 0.20 for the recessive model were used. Marker allele frequencies were generated from over 150 unrelated Caucasian individuals.). Multipoint non-parametric lod scores (LOD*) were calculated using GENEHUNTER-PLUS (A. Kong, N. J. Cox, Am. J. Hum Genet. 61, 1179 (1997); Sex-averaged intermarker distances from the Marshfield Center for Medical Genetics genetic linkage maps (http://research/marshfieldclinic.org/genetics/) were used in these analyses. In contrast to non-parametric linkage approaches which consider allele sharing in pairs of affected siblings [N. Risch, Am. J. Hum. Genet. 46, 222 (1990)], GENEHUNTER-PLUS considers allele sharing across pairs of affected relatives (or all affected relatives in a family) in moderately sized pedigrees. We selected GENEHUNTER-PLUS to take advantage of the additional power contributed to the sample by the 75 affected relative pairs that would be ignored by an affected sibpair analysis. Due to computational constraints on pedigree size, 27 unaffected individuals from 12 families were omitted from GENEHUNTER-PLUS analysis).

Due to the potential genetic heterogeneity in this sample, a priori we stratified the data set in two ways. The first was to divide the sample by age at onset. Families with at least one member with early-onset (<40 years (L. I. Golbe, Neurology 41, 168 (1991))) PD (n=18) were considered separately from the rest of the (Gate-onset) families (n=156) (Mean age at onset in the early-onset families was 39.7 years (range: 12-66), while mean age at onset in the late-onset families was 62.7 years (range: 40-90). The two age of onset groups were similar with respect to average family size and structure.). As well, nine families (all late-onset) contained at least one affected individual who were determined to be non-responsive to levodopa therapy; these families were considered separately from the rest of the late-onset families (n=147).

The intent of an initial complete genomic screen is to identify regions of the genome likely harboring susceptibility loci for more thorough analysis. Because genetic heterogeneity likely reduces the power to detect statistically significant evidence of linkage using the traditional criterion of a lod score >3, we chose a more liberal criterion of a lod score >1 in the overall sample for consideration of a region as interesting and warranting initial follow-up. Regions were then prioritized into two groups for efficient laboratory analysis: regions generating lod scores >1 on both two-point and multipoint analyses were classified as priority 1, while regions with lod scores >1 on only one test were designated priority 2. While this approach may increase the number of false-positive results that are examined in more detail, it decreases the more serious (in this case) false-negative rate.

B. Results

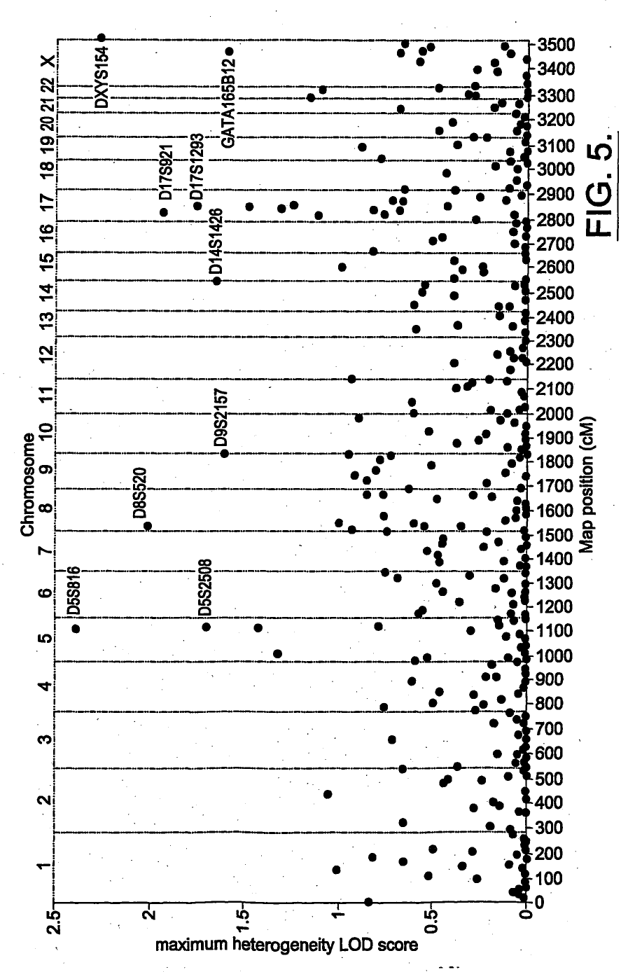

Plots of the two-point MLOD, overall and stratified by age at onset, are presented in FIGS. 5-7. Genetic regions generating LOD*>1 are listed in Table 5. Markers on chromosomes 5p, 5q, 8p, 9q, 14q, 17q, and Xq generated interesting two-point lod scores (MLOD >1) in the overall sample of 174 families (FIG. 5). Four of these regions also produced multipoint LOD* scores >1 and were classified as priority 1 for follow-up. The strongest evidence for linkage in the overall data set was on chromosome 8p (MLOD=2.01 at D8S520; LOD*=2.22). Other regions with interesting two-point and multipoint results were 5q (MLOD=2.39 at D5S816; LOD*=1.5), 17q (MLOD=1.92 at D17S921; LOD*=2.02), and 9q (MLOD=1.59 at D9S2157; LOD*=1.47). Three regions with two-point lod scores >1 (5p, 14q, Xq) did not have multipoint LOD*>1 and were designated priority 2 for follow-up.

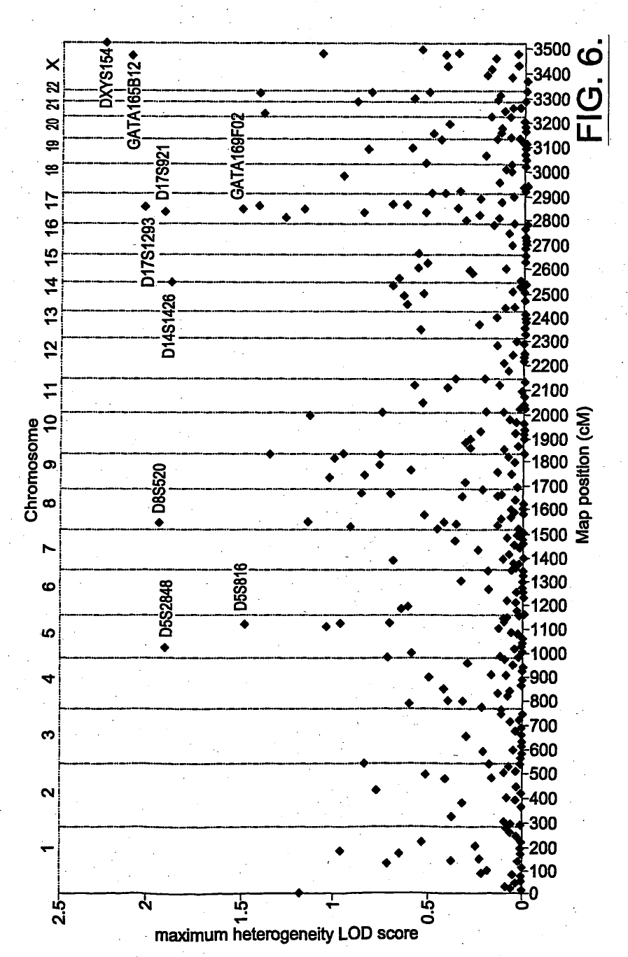

Two-point results obtained from the subset of 156 late-onset families (FIG. 6) were essentially similar. In addition to the seven interesting regions identified in the overall sample, lod scores were >1 at markers on chromosomes 21p and 22q. The strongest result in this subset was on 17q (MLOD=2.05 at D17S1293; LOD*=2.31), followed by 8p (MLOD=1.96 at D8S520; LOD*=1.92), and 9q (MLOD=1.36; LOD*=1.4). The other six regions with interesting two-point results (5p, 5q, 14q, 21p, 22q, and Xq) generated multipoint LOD*<1.

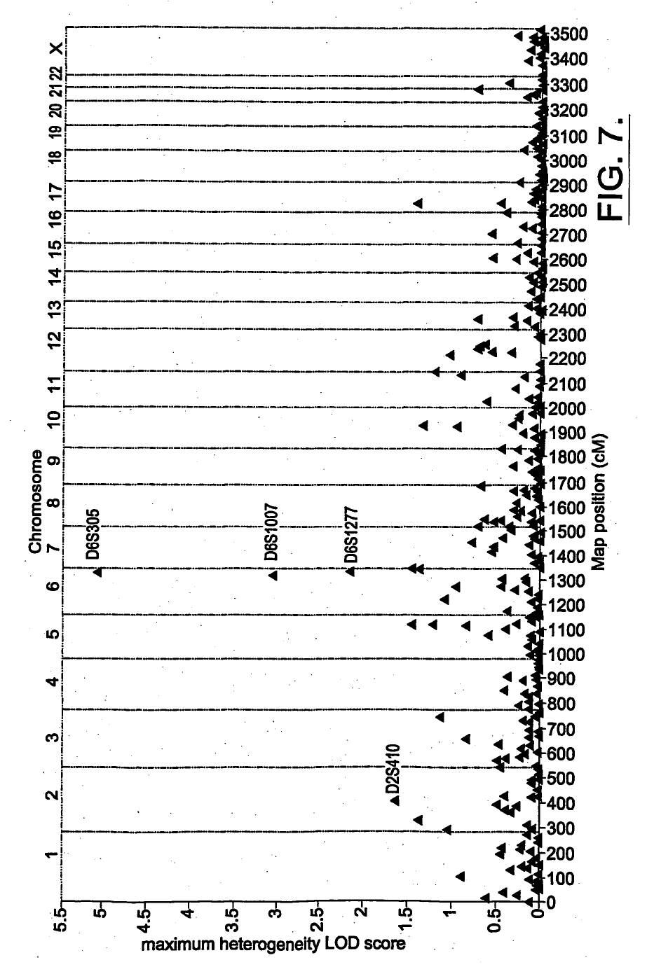

In the subset of 18 early-onset families (FIG. 7), only two regions identified in the overall sample (5q and 17q) generated interesting two-point results. Five additional regions (2q, 6q, 10q, 11 q, and 12q) generated lod scores >1 in this subset. A highly significant result was obtained at D6S305 (MLOD=5.07; LOD*=5.47). An additional region with interesting two-point and multipoint results was identified on chromosome 11q (MLOD=1.22 at D11S4131; LOD*=1.53). Multipoint LOD* scores on chromosomes 2q, 5q, 10q, 12q, and 17q were less significant (LOD*<1).

Examination of the nine families containing affected individuals whose PD was not responsive to levodopa therapy produced several novel results. In addition to supporting linkage to regions on chromosomes 5q, 9q, 17q, and 22q indicated by the overall late-onset subset, these nine families also implicated regions on chromosomes 3q, 6q, 20p, and a second region on 9q. The strongest results in this subset were obtained from the multipoint analysis of chromosome 9q (MLOD=0.98 at D9S2157; LOD*=2.59). Analysis of the 147 remaining late-onset families separately did not generate any significantly different two-point results from the analysis of all 156 late-onset families.

TABLE 4
Composition of the data set: Number of Affected Relative Pairs*

| Mean number of sampled affected members | 2.3 ± 0.6 (range: 2-6) |
|---|---|
| per family | |
| Mean number of sampled affected relative pairs | 1.5 ± 1.4 (range 1-15) |
| per family | |
| Number of sampled affected sibpairs | 185 |
| Number of sampled affected avuncular pairs | 19 |
| Number of sampled affected cousin pairs | 51 |
| Number of sampled affected parent-child pairs | 5 |
| Total number of affected relative pairs | 260 |

*all possible affected relative pairs counted

TABLE 5
Regions generating multipoint LOD* greater than 1.

| Chro- | | peak | Two-point | | Multipoint | |
|---|---|---|---|---|---|---|
| mosome | Set | marker | MLOD | location | Peak LOD* | location |
| 3q | NLDR | D3S2460 | 1.62 | 135 | 1.54 | 134 |
| 5q | ALL | D5S816 | 2.39 | 139 | 1.5 | 139 |
| | NLDR | D5S820 | 1.47 | 160 | 1.04 | 153 |
| 6q | EOPD | D6S305 | 5.07 | 166 | 5.47 | 166 |
| 8p | ALL | D8S520 | 2.01 | 21 | 2.22 | 27 |
| | LOPD | D8S520 | 1.96 | 21 | 1.92 | 27 |
| 9q | NLDR | D9S301 | 1.52 | 66 | 1.01 | 66 |
| 9q | ALL | D9S2157 | 1.59 | 147 | 1.47 | 147 |
| | LOPD | D9S2157 | 1.36 | 147 | 1.4 | 145 |
| | NLDR | D9S2157 | 0.98 | 147 | 2.59 | 140 |
| 11q | EOPD | D11S4131 | 1.22 | 139 | 1.53 | 139 |
| 17q | ALL | D17S921 | 1.92 | 36 | 2.02 | 56 |
| | LOPD | D17S1293 | 2.05 | 56 | 2.31 | 56 |
| | NLDR | D17S1843 | 2.52 | 41 | 1.26 | 36 |

EOPD = early-onset PD;
LOPD = late-onset PD;
NLDR = non-levodopa-responsive PD

In summary, these results provide very strong evidence that several genes influence the development of familial PD, and that age at onset and levodopa response pattern influence the evidence for linkage to each gene. In contrast to recent contentions that most late-onset PD is caused by environmental factors (C. M. Tanner et al., JAMA 281, 341 (1999)), these data suggest that several genes may influence the development of late-onset familial PD. Identification of one or more of these genes underlying late-onset familial PD will provide invaluable insight into the pathogenesis and potential treatment of PD.

### EXAMPLE 5

### Association of Tau with Late-Onset Parkinson Disease

To examine the role of the tau gene in PD, five polymorphisms in the tau gene were tested for association with PD in a sample of PD families.

A. Methods

Study Subjects. The sample consists of 1056 individuals in 235 families (N=17). Most families in this study are discordant sibships (at least one affected and one unaffected sibling) without parental samples (N=156). A smaller number are nuclear families with at least one affected individual with both parents (N=40) or only one parent (N=3) sampled. The remaining families are more complex containing more than a single nuclear family or sibship (N=36). This data set contains many of the families used in the our PD genomic screen (see above) and some additional families. Only families with at least one affected individual with either both parents sampled or at least one unaffected sibling sampled were included to provide more flexibility in the association analyses. When possible, unaffected siblings who were older at age of exam than the age of onset of their affected siblings were sampled. The mean age of onset in affected individuals in the sample is 57.5 years, and the mean age of unaffected individuals is 66.8 years (Age at onset was self-reported, defined as the age at which the affected individual could first recall noticing one of the cardinal signs of PD).

Excluded from participation are individuals with a history of encephalitis, neuroleptic therapy within the year prior to diagnosis, evidence of normal pressure hydrocephalus, or a clinical course with unusual features, suggestive of atypical or secondary parkinsonism. To exclude PSP, FTDP, and other parkinsonian conditions from the PD affected group, all subjects in the PD affected group had to meet strict clinical criteria (All subjects affected with PD in this study had asymmetric motor symptoms at onset, no postural instability with falls early in the disease course, and no supranuclear down- or lateral-gaze palsy. The presence of any one of these exclusion criteria was sufficient to prevent inclusion in the PD affected group, and excluded subjects with clinical features of PSP and other atypical parkinsonian syndromes. Subjects with FTDP were excluded from the PD affected group by clinical criteria requiring the absence of dementia at onset and the presence of asymmetric onset of motor symptoms. Other parkinsonian syndromes were screened by additional clinical criteria such as absence of severe autonomic neuropathy or signs of significant cerebellar dysfunction (multiple system atrophy, MSA); absence of abrupt symptom onset or of a stepwise course (vascular parkinsonism); and absence of unilateral dystonia with apraxia or cortical sensory loss (cortical-basal ganglionic degeneration, CBGD)).

Family history of PD was documented for each family by conducting a standard interview with the proband or a knowledgeable family informant The results of this interview were used to generate pedigrees documenting the extent of family history of PD out to three degrees of relationship (1st cousins).

Molecular Analysis. Five SNPs in tau, previously tested by Baker et al. for association with PSP (M. Baker, et al, Hum. Mol. Genet. 8, 711 (1999)), were chosen for analysis of association in the PD family sample. Two SNPs are intronic: one in intron 3 (SNP 3) and one in intron 11 (SNP 11). The other three SNPs chosen are all in exon 9 (SNPs 9i, 9ii, 9iii). The dinucleotide repeat polymorphism between exons 9 and 10 was also tested (C. Condad, et al, Ann Neurol. 41, 277 (1997)).

DNA was extracted from whole blood using Puregene kits (Gentra Systems, Minneapolis, Minn.) by the Center for Human Genetics' DNAbanking Core. SNPs were genotyped using a modification of the gel-based Oligonucleotide Ligation Assay (OLA) (F. A Eggerding, D. M. Iovannisci E. Brinson, P. Grossman, E. S. Winn-Deen, Hum. Mutat. 5, 153 (1995)) which consists of an initial multiplex PCR amplification followed by a subsequent ligation (PCR amplification was performed in 10 uL reactions (30 ng DNA, 1× Gibco PCR buffer, 0.6 mM dNTP, 3.0 mM Mg, 0.5U Gibco Platinum Taq and 0.04 μg forward and reverse primers) using MJ PTC200 or Prius96Plus (MWG-Biotech, Ebersberg, Germany) thermocyclers for 40 cycles (94° C. 4 min.; 5×[94° C./30 sec., 55° C./30 sec, 72° C./30 sec]; 20×[94° C./5 sec., 55° C./30 sec, 72° C./45 sec]; 15×[94° C./5 sec., 55° C./45 sec, 72° C./80 sec]; 72° C./7 min) followed by a 30 minute incubation at 94C to heat kill the enzyme. Two microliters of the PCR reaction mix were transferred and dried prior to being resuspended in 10 μL of Ligation mix [1×Taq DNA ligase buffer, 4U Taq DNA thermostable ligase] (New England BioLabs, Beverly, Mass.). Allele specific probes were fluorescently labeled using Fam or Cy3 and common probes were phosphorylated on the 5′ end. Ligations were performed in a MJ PTC200 or Primus96Plus thermocycler (40×[94° C., 20 sec; 50° C., 1 min]). Reactions were stopped with the addition of 20 ul of loading/stop dye (98% deionized formamide, 10 mM EDTA, 0.025% xylene cyanol, 0.025% bromophenol blue). Approximately 4 ul of each sample was loaded onto a 6% polyacrylamide gel, run for approximately 40 minutes, and scanned on a Hitachi FMBio II fluorescent static scanner. Images were analyzed using BioImage software. Genotyping of the microsatellite marker was performed by fluorescence imaging using the FASST method previously described. [J. M: Vance, K. Ben Othmane, Methods of Genotyping, J. L Haines and M Pericak-Vance, Eds. (John Wiley & Sons, Inc., New York, 1998]. To ensure correct OLA genotyping, representative OLA genotypes were checked for accuracy using sequencing (CEQ2000XL)). Table 6 gives PCR primers and OLA probes for SNPs used in this study.

Quality control was conducted by the Center for Human Genetics' Data Coordinating Center (DCC) using a set of internal QC samples to which the technicians were blinded (J. Rimmler, et al, Am. Soc. Hum. Gem 63, A240 (1998)). As an additional level of QC for our candidate gene analyses, each pair of markers within each gene was tested for recombination using Fastlin (R. W. Cottingham et al., Am. J Hum. Gen. 53, 252 (1993); A. Schaffer et al., Hum. Hered 44, 225 (1994)). All individuals in families showing evidence of recombination between markers were checked for genotype misreads. Because four of these SNPs have been reported elsewhere (M. Baker, et al, Hum. Mol. Genet. 8, 711 (1999)) to be in strong linkage disequilibrium, genotypes of individuals showing evidence of haplotypes that were not expected were also checked. In each case, rereads or direct sequencing resolved the recombination or haplotype discrepancy.

Statistical Analysis. Two complementary methods for association analysis that are appropriate for this family data were used: (1) the pedigree disequilibrium test (PDT) (E. Martin et al., Am. J. Hum. Genet. 67, 146 (2000)), and (2) the likelihood ratio test (LRT) implemented in the program Transmit (D. Clayton, Am. J. Hum. Gen. 65, 1170 (1999). A version of the PDT based on the PDT-sum statistic was used (E. Martin et al., Am. J. Hum. Gen. 68, 1065-1067 (2001)). The robust variance estimator was used in the LRT of Transmit to assure validity as a test of association in sibships of arbitrary size. The data set used for association analyses consists of few extended pedigrees, thus the Transmit analysis is reported based on all nuclear families. P-values for a global test of significance were computed using the chi-squared distribution with h-1 degrees of freedom, where h is the number of distinct haplotypes observed (h=2 for single-locus tests). SNPs were analyzed individually using both methods. Haplotype analysis was also conducted, testing for association with haplotypes including multiple SNPs, using Transmit (Inferred haplotypes with frequencies <0.01 were combined with more frequent haplotypes).

To further refine the analyses, two criteria were considered for stratification. Families were classified as family-history positive if a relative of the proband is reported to be affected with PD, or family-history negative if there was no report of PD in the family other than the proband. Families were classified as early-onset if there was at least on individual with age of onset <40 years and late-onset if all individuals had age of onset ≧40 years. Nine of the early-onset families have known mutations in the parkin gene. To try to improve homogeneity in the sample, the early-onset families excluding those with known parkin mutations were also analyzed. The PDT and Transmit test were conducted using families within each stratum.

A single affected and unaffected individual were selected at random from each family for tests of Hardy-Weinberg disequilibrium (HWD) and linkage disequilibrium between markers. Analysis was conducted in the affected sample and unaffected sample separately. The tests implemented in the Genetic Data Analysis Program (version 1.0 d16b) were used (P. O. Lewis, D. Zaykin, Genetic Data Analysis: Computer program for analysis of allelic data. 1.0(d15) (2000)). P-values were estimated using 3200 permutations.

B. Results

Table 7 shows p-values for single-locus association analyses using PDT and Transmit. The Transmit test was significant (p<0.05) for three of the markers (SNPs 3, 9i and 11). The PDT shows the same trend as the Transmit tests, giving marginally significant results at the same markers. For each marker, it is the more common allele (allele 2) that is positively associated with PD in our sample. Maximum likelihood estimates for allele frequencies of the positively associated allele, from Transmit, are shown in Table 7. For PDT, the positively associated allele occurs more frequently in affected siblings than in unaffected siblings. For Transmit, the positively associated allele is transmitted from parents to affected individuals more frequently than expected. For each marker, PDT and Transmit both show the same allele to be positively associated. The high frequency of the allele at SNP 9iii (Table 7) offers an explanation for why no association was detected. If the positively-associated allele is at high frequency in the population, it will be difficult to detect the association since there cannot be a large difference between the allele frequency in the population and in the affecteds, even if the allele has frequency 1.0 in the affecteds.

As has been reported elsewhere (M. Baker, et al, Hum. Mol. Genet. 8, 711(1999)), there was considerable linkage disequilibrium between the markers. In all individuals, the two haplotypes H1 and H2 observed by Baker et al were the only haplotypes directly observed for SNPs 3, 9i, 9ii and 11. There was no evidence of the existence of other haplotypes for these four markers in our sample. P-values smaller than 1/3200 were estimated for all combinations of these markers. For SNP 9iii, the rare allele occurs almost exclusively with common haplotype, suggesting other haplotypes are old and this allele at 9iii arose more recently on the common H1 haplotype. Significant linkage disequilibrium was not detected between SNP 9iii and the other four markers in neither the affected nor the unaffected samples. No evidence for deviation from Hardy-Weinberg equilibrium was found in affecteds or unaffecteds for any of the markers.

Table 8 shows the results of the haplotype association analysis with Transmit for the five-locus haplotypes. Only 3 common haplotypes were observed for the five loci. Individual p-values for the two most common haplotypes were significant with p<0.01. The haplotype carrying alleles 11121 (at SNPs 3, 9i, 9ii, 9iii and 11, respectively) is significantly under-transmitted to affected individuals, while the haplotype carrying alleles 22222 is significantly over-transmitted to affected individuals. Interestingly, the 22222 haplotype corresponds to the H1 haplotype previously associated with PSP (Baker et al., supra). There is no evidence for association with the H1 sub-haplotype carrying allele 1 at 9iii, suggesting that the putative susceptibility allele may occur with increased frequency on the H1-haplotype carrying allele 2 at 9iii.

Table 9 shows results for stratified analyses using Transmit. The single-locus and haplotype association tests in family-history-positive families are close to the p-values in the overall sample. The tests in family-history-negative families are not significant for any of the comparisons. The level of significance tends to decrease in the early- and late-onset families relative to the whole sample, however the results in the late-onset subset are marginally significant (p<0.1) for three of the SNPs and the five locus haplotype. In general, significance decreased for tests in the early-onset families when families with known parkin, mutations were excluded (results not shown). However, this subset contains only 30 families, thus it would be quite difficult to detect an association, even if the sample is more homogeneous.

A dinucleotide repeat polymorphism, previously associated with PSP (Baker et al., supra), lying between exons 9 and 10 in the tau gene, was also examined for association with PD. The repeat was typed in a set of. 249 multiplex PD families, ascertained for family-based linkage studies as described above, which overlaps with the data set used for SNP analyses. A significant association was found with the LRT of Transmit (global test p=0.0286), with the common allele, a0, being significantly overtransmitted to affected individuals and allele a3 being significantly undertransmitted. These results are consistent with the findings of Baker et al., supra for PSP, though not as significant, and further supports the recent report by Pastor et al. of a difference in a0 allelic frequency between PD patients and controls (P. Pastor, et al, Neurol 47, 242 (2000)).

TABLE 6
PCR primers and OLA probes for SNPs used in association analyses.

| | | |
|---|---|---|
| SNP | PCR primer | OLA probe |

| | | | | | | |
|---|---|---|---|---|---|---|
| 3 | IVS3 + 9A > G | forward | gggctgctttctggcatatg | Allele 1 | G | 5′-Cy3-aggaaccacaggtgagggtg |
| | | | (SEQ ID NO:5) | | | (SEQ ID NO:6) |
| | | reverse | cctcacttctgtcacaggtc | Allele 2 | A | 5′-Cy3-agaaggaaccacaggtgagggta |
| | | | (SEQ ID NO:7) | | | (SEQ ID NO:8) |
| | | | | common | | 5′-Pho-agccccagagacccccaggcagtc |
| | | | | | | (SEQ ID NO:9) |
| 9i | c1632A > G | forward | ccacccgggagcccaagaaggtgcc | Allele 1 | G | 5′-Fam-gggagcccaagaaggtggcg |
| | | | (SEQ ID NO:10) | | | (SEQ ID NO:11) |
| | Ala544Ala | reverse | ctggtgcttcaggttctcagtg | Allele 2 | A | 5′-Fam-cccgggagcccaagaaggtggca |
| | | | (SEQ ID NO:12) | | | (SEQ ID NO:13) |
| | | | | common | | 5′-Pho-gtggtccgtactccacccaagtcgccgtc |
| | | | | | | ttccgc |
| | | | | | | (SEQ ID NO:14) |
| 9ii | c1716T > C | forward | cgagtcctggcttcactcc | Allele 1 | C | 5′-Cy3-ccatgccagacctgaagaac |
| | | | (SEQ ID NO:15) | | | (SEQ ID NO:16) |
| | Asn572Asn | reverse | cttccaggcacagccatacc | Allele 2 | T | 5′-Cy3-tgcccatgccagacctgaagaat |
| | | | (SEQ ID NO:17) | | | (SEQ ID NO:18) |
| | | | | common | | 5′-Pho-gtcaagtccaagatcggctccactgaga |
| | | | | | | (SEQ ID NO:19) |
| 9iii | c1761G > A | forward | cgagtcctggcttcactcc | Allele 1 | A | 5′-Fam-agaacctgaagcaccagcca |
| | | | (SEQ ID NO:20) | | | (SEQ ID NO:21) |
| | Pro587Pro | reverse | cttccaggcacagccatacc | Allele 2 | G | 5′-Fam-ctgagaacctgaagcaccagccg |
| | | | (SEQ ID NO:22) | | | (SEQ ID NO:23) |
| | | | | common | | 5′-Pho-ggaggcgggaaggtgagagtggctgg |
| | | | | | | (SEQ ID NO:24) |
| 11 | IVS11 + 34G > A | forward | gctcattctctctcctcctc | Allele 1 | A | 5′-Cy3-ggtgagggttgggacgggaa |
| | | | (SEQ ID NO:25) | | | (SEQ ID NO:26) |
| | | reverse | ccaggactcctccaccccatgcagc | Allele 2 | G | 5′-Cy3-gaaggtgagggttgggacgggag |
| | | | (SEQ ID NO:27) | | | (SEQ ID NO:28) |
| | | | | common | | 5′-Pho-ggtgcagggggtggaggagtcctggtgag |
| | | | | | | gctggaac |
| | | | | | | (SEQ ID NO:29) |

TABLE 7
P-values for PDT and Transmit single-locus tests.

| | MLE's for Allele | | |
|---|---|---|---|
| SNP | Frequencies¹ | PDT² | Transmit² |
| 3 | 0.794 | 0.062 | 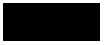 |
| 9i | 0.793 | 0.076 | 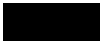 |
| 9ii | 0.790 | 0.113 | 0.106 |
| 9iii | 0.955 | 0.638 | 0.866 |
| 11 | 0.793 | 0.055 | 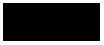 |

¹For positively associated allele
²P-values from cu-squared distribution
Note:
P-values < 0.05 are highlighted.

TABLE 8
P-values for Transmit tests for five-locus SNP haplotypes.

| Haplotype for | |
|---|---|
| 3/9i/9ii/9iii/11 | P-values |
| 11121 | 0.007 |
| 22212 | 0.863 |
| 22222 | 0.009 |
| Global Test | 0.024 |

Note:
Individual haplotype tests are compared to a chi-square distribution with 1 df. Global test is compared to chi-square distribution with 2 df.

TABLE 9
P-values for single-locus and 5-locus haplotype Transmit
tests in stratified data sets.

| | Family-history | Family-history | Early | Late |
|---|---|---|---|---|
| | positive | negative | onset | onset |
| SNPs | (N = 181) | (N = 54) | (N = 39) | (N = 196) |
| 3 | 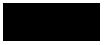 | 0.957 | 0.076 | 0.076 |
| 9i | 0.055 | 0.645 | 0.682 | 0.059 |
| 9ii | 0.128 | 0.585 | 0.534 | 0.149 |
| 9iii | 0.707 | 0.170 | 0.076 | 0.816 |
| 11 | 0.055 | 0.524 | 0.199 | 0.095 |
| Haplotype for | 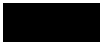 | 0.479 | 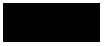 | 0.093 |
| 3/9i/9ii/9iii/11 | | | | |

Note:
P-values <0.05 are highlighted. N is the number of families in the stratum.

The foregoing is illustrative of the present invention, and is not to be construed as limiting thereof. The invention is defined by the following claims, with equivalents of the claims to be included therein.

That which is claimed is:

1. A method of screening a subject for Parkinson's disease, comprising the steps of:
detecting the presence or absence of a functional polymorphism associated with a gene linked to Parkinson's disease;
the presence of said functional polymorphism indicating said subject is afflicted with or at risk of developing Parkinson's disease;
said gene selected from the group consisting of the synphilin gene and the ubiquitin conjugating enzyme (UBE2B) gene on chromosome 5, the Parkin gene on chromosome 6, the NAT1 gene and NAT2 gene on chromosome 8, the proteasome subunits Z and S5 genes and the Torsin A or Torsin B genes on chromosome 9, and the ubiquitin B gene and Tau genes on chromosome 17.

2. The method according to claim 1, wherein said Parkinson's disease is late-onset Parkinson's disease.

3. The method according to claim 1, wherein said subject has previously been determined to be at risk for Parkinson's disease.

4. The method according to claim 1, wherein said method is a prognostic method.

5. The method according to claim 1, wherein said method is a diagnostic method.

6. The method according to claim 1, wherein said functional polymorphism is in said gene.

7. The method according to claim 1, wherein said functional polymorphism is a deletion mutation.

8. The method according to claim 1, wherein said functional polymorphism is a substitution mutation.

9. The method according to claim 1, wherein said detecting step is carried out by:
collecting a biological sample from said subject; and then
detecting the presence or absence of said mutation from said biological sample.

10. The method according to claim 1, wherein said gene is selected from the group consisting of the synphilin gene and the ubiquitin conjugating enzyme (UBE2B) gene on chromosome 5.

11. A method of screening a subject for Parkinson's disease, comprising the steps of:
detecting the presence or absence of a Parkin gene exon 3 deletion mutation in said subject;
the presence of said deletion mutation indicating said subject is afflicted with or at risk of developing Parkinson's disease.

12. The method according to claim 11, wherein said deletion mutation includes a deletion within base pairs 438-477.

13. The method according to claim 11, wherein said deletion mutation is a deletion of base pairs 438 through 477, inclusive.

14. The method according to claim 11, wherein said Parkinson's disease is late-onset Parkinson's disease.

15. The method according to claim 11, wherein said subject has previously been determined to be at risk for Parkinson's disease.

16. The method according to claim 11, wherein said method is a prognostic method.

17. The method according to claim 11, wherein said method is a diagnostic method.

18. The method according to claim 11, wherein said detecting step is carried out by:
collecting a biological sample from said subject; and then
detecting the presence or absence of said mutation from said biological sample.

19. The method according to claim 11, wherein said detecting step comprises a polymerase chain reaction amplification step.

20. A method of screening a subject for Parkinson's disease, comprising the steps of:
detecting the presence or absence of a functional polymorphism associated with a gene linked to Parkinson's disease;
the presence of said functional polymorphism indicating said subject is afflicted with or at risk of developing Parkinson's disease;
wherein said gene is the Parkin gene on chromosome 6.

21. The method according to claim 20, wherein said Parkinson's disease is late-onset Parkinson's disease.

22. The method according to claim 20, wherein said subject has previously been determined to be at risk for Parkinson's disease.

23. The method according to claim 20, wherein said method is a prognostic method.

24. The method according to claim 20, wherein said method is a diagnostic method.

25. The method according to claim 20, wherein said functional polymorphism is in said gene.

26. The method according to claim 20, wherein said functional polymorphism is a substitution mutation.

27. The method according to claim 20, wherein said detecting step is carried out by:
collecting a biological sample from said subject; and then
detecting the presence or absence of said mutation from said biological sample.

28. The method according to claim 20, wherein said functional polymorphism comprises a G to A substitution at position 1390.

29. The method according to claim 20, wherein said functional polymorphism comprises a C to T substitution at position 924.

30. The method according to claim 20, wherein said functional polymorphism comprises a C to T substitution at position 1412.

31. The method according to claim 20, wherein said functional polymorphism comprises a G to A substitution at position 859.

32. The method according to claim 20, wherein said functional polymorphism comprises a deletion of AG at positions 202 to 203.

33. The method according to claim 20, wherein said functional polymorphism comprises a G to A substitution at position 199.

34. The method according to claim 20, wherein said functional polymorphism comprises a G to T substitution at 4 base pairs past the end of exon 9.

35. The method according to claim 20, wherein said functional polymorphism comprises a C to A substitution at position 346.

36. The method according to claim 20, wherein said functional polymorphism comprises a G to A substitution at position 885.

37. A method of screening for susceptibility to Parkinson's Disease in a subject, the method comprising:
determining the presence or absence of an allele of a polymorphic marker in the DNA of the patient, wherein (i) the allele is associated with the phenotype of Parkinson's disease, and wherein (ii) the polymorphic marker is within a segment selected from the group consisting of:
a segment of chromosome 2 bordered by D2S2982 and D2S1240;
a segment of chromosome 2 bordered by D2S1400 and D2S2291;
a segment of chromosome 2 bordered by D2S2161 and D2S1334;
a segment of chromosome 2 bordered by D2S161 and D2S2297;
a segment of chromosome 3 bordered by D3S1554 and D3S3631;
a segment of chromosome 3 bordered by D2S1251 and D3S3546;
a segment of chromosome 5 bordered by D5S2064 and D5S1968;
a segment of chromosome 5 bordered by D5S2027 and D5S1499;
a segment of chromosome 5 bordered by D5S816 and D5S1960;
a segment of chromosome 6 bordered by D6S1703 and D6S1027;
a segment of chromosome 6 bordered by b6S1581 and D6S2522;
a segment of chromosome 8 bordered by D8S504 and D8S258;
a segment of chromosome 9 bordered by D9S259 and D9S776;
a segment of chromosome 9 bordered by D9S1811 and D9S2168;
a segment of chromosome 10 bordered by D10S1122 and D10S1755;
a segment of chromosome 11 bordered by D11S4132 and D11 S4112;
a segment of chromosome 12 bordered by D12S1042 and D12S64;
a segment of chromosome 14 bordered by D14S291 and D14S544;
a segment of chromosome 17 bordered by D17S1854 and D17S1293;
a segment of chromosome 17 bordered by D17S921 and D17S669;
a segment of chromosome 21 bordered by D21S1911 and D21S1895;
a segment of chromosome 22 bordered by D22S425 and D22S928;
a segment of chromosome X bordered by DXS6797 and DXS1205; and
a segment of chromosome X bordered by DXS9908 and X telomere;
the presence of said allele indicating said subject is at risk of developing Parkinson's disease.

38. The method according to claim 37, wherein said Parkinson's disease is late-onset Parkinson's disease.

39. The method according to claim 37, wherein said subject has previously been determined to be at risk for Parkinson's disease.

40. The method according to claim 37, wherein said method is a prognostic method.

41. The method according to claim 37, wherein said method is a diagnostic method.

42. A method of screening a subject for Parkinson's disease, comprising the steps of:
detecting the presence or absence of a functional polymorphism associated with a gene linked to Parkinson's disease;
the presence of said functional polymorphism indicating said subject is afflicted with or at risk of developing Parkinson's disease;
wherein said gene is the tau gene on chromosome 17.

43. The method according to claim 42, wherein said functional polymorphism is IVS3+9A>G.

44. The method according to claim 42, wherein said functional polymorphism is c1632A>G.

45. The method according to claim 42, wherein said functional polymorphism is c1716T>C.

46. The method according to claim 42, wherein said functional polymorphism is c1761G>A.

47. The method according to claim 42, wherein said functional polymorphism is IVS11+34G>A.

48. The method according to claim 42, wherein said Parkinson's disease is late-onset Parkinson's disease.

49. The method according to claim 42, wherein said subject has previously been determined to be at risk for Parkinson's disease.

50. The method according to claim 42, wherein said method is a prognostic method.

51. The method according to claim 42, wherein said method is a diagnostic method.

52. A computer assisted method of identifying a proposed treatment for Parkinson's disease, comprising the computer assisted steps of:
(a) storing a database of biological data for a plurality of patients, the biological data including for each of said plurality of patients (i) a treatment type, (ii) at least one genetic marker associated with Parkinson's disease, and (iii) at least one disease progression measure for Parkinson's disease from which treatment efficacy may be determined; and then
(b) querying said database to determine the dependence on said genetic marker of the effectiveness of a treatment type in treating Parkinson's disease, to thereby identify a proposed treatment as an effective treatment for a patient carrying a particular marker for Parkinson's disease.

53. The method according to claim 52, wherein said marker is a functional polymorphism associated with a gene linked to Parkinson's disease;
said gene selected from the group consisting of the synphilin gene and the ubiquitin conjugating enzyme (UBE2B) gene on chromosome 5, the Parkin gene on chromosome 6, the NAT1 gene and NAT2 gene on chromosome 8, the proteasome subunits Z and S5 genes and the Torsin A or Torsin B genes on chromosome 9, and the ubiquitin B gene and Tau genes on chromosome 17.

54. The method according to claim 52, wherein said marker is a Parkin gene exon 3 deletion mutation.

55. The method according to claim 52, wherein said marker is the presence or absence of an allele of a polymorphic marker in the DNA of the patient, wherein (i) the allele is associated with the phenotype of Parkinson's disease, and wherein (ii) the polymorphic marker is within a segment selected from the group consisting of:
a segment of chromosome 2 bordered by D2S2982 and D2S1240;
a segment of chromosome 2 bordered by D2S1400 and D2S2291;
a segment of chromosome 2 bordered by D2S2161 and D2S1334;
a segment of chromosome 2 bordered by D2S161 and D2S2297;
a segment of chromosome 3 bordered by D3S1554 and D3S3631;
a segment of chromosome 3 bordered by D2S1251 and D3S3546;
a segment of chromosome 5 bordered by D5S2064 and D5S1968;
a segment of chromosome 5 bordered by D5S2027 and D5S1499;
a segment of chromosome 5 bordered by D5S816 and D5S1960;
a segment of chromosome 6 bordered by D6S1703 and D6S1627;
a segment of chromosome 6 bordered by D6S1581 and D6S2522;
a segment of chromosome 8 bordered by D8S504 and D8S258;
a segment of chromosome 9 bordered by D9S259 and D9S776;
a segment of chromosome 9 bordered by D9S1811 and D9S2168;
a segment of chromosome 10 bordered by D10S1122 and D10S1755;
a segment of chromosome 11 bordered by D11S4132 and D11S4112;
a segment of chromosome 12 bordered by D12S1042 and D12S64;
a segment of chromosome 14 bordered by D14S291 and D14S544;
a segment of chromosome 17 bordered by D17S1854 and D17S1293;
a segment of chromosome 17 bordered by D17S921 and D17S669;
a segment of chromosome 21 bordered by D21S1911 and D21S1895;
a segment of chromosome 22 bordered by D22S425 and D22S928;
a segment of chromosome X bordered by DXS6797 and DXS1205; and
a segment of chromosome X bordered by DXS9908 and X telomere;
the presence of said allele indicating said subject is at risk of developing Parkinson's 25 disease.

56. The method according to claim 52, wherein said marker is a functional polymorphism associated with a gene linked to Parkinson's disease,
wherein said gene is the tau gene on chromosome 17.

57. The method according to claim 52, wherein treatment type is selected from the group consisting of control treatments and experimental treatments.

58. The method according to claim 57, wherein said database includes a plurality of patients having control treatments and a plurality of patients having experimental treatments.

59. The method according to claim 57, wherein said control treatment is selected from the group consisting of placebo treatments and treatments with a known treatment for Parkinson's disease.

60. The method according to claim 57, wherein said database includes a plurality of patients having control treatment with a placebo, a plurality of patients having control treatments with a known treatment for Parkinson's disease, and a plurality of patients having experimental treatments.

61. The method according to claim 57, wherein said at least one disease progression measure is selected from the group consisting of tremor measures, rigidity measures, and akinesia measures.

62. The method according to claim 57, wherein said biological data for each of said plurality of patients includes at least three distinct genetic markers associated with Parkinson's disease.

63. The method according to claim 57, wherein said biological data for each of said plurality of patients includes at least five distinct genetic markers associated with Parkinson's disease.

64. The method according to claim 57, wherein said biological data for each of said plurality of patients includes at least ten distinct genetic markers associated with Parkinson's disease.

65. A method of treating a subject for Parkinson's disease, comprising the steps of:
determining the presence of a preselected marker for Parkinson's disease in said subject; and then
administering to said subject a treatment effective for treating Parkinson's disease in a subject that carries said marker,
and wherein said treatment is identified by the method of claim 52.

66. The method according to claim 65, wherein said Parkinson's disease is late-onset Parkinson's disease.


SEQUENCE LISTING (29 sequences)

SEQ ID NO: 1 - DNA, 60 bp, Homo sapiens
  misc_feature (1)..(60): Exon 3 from Parkin gene from individuals with Parkinson′s Disease
actcgggtggacctcagcagctcagtcctcacagcaggaaggactcaccaccagctggaa

SEQ ID NO: 2 - DNA, 100 bp, Homo sapiens
  misc_feature (1)..(100): Exon 3 of Parkin gene from control individual
actcgggtggacctcagcagctcagtcctcccaggagactctgtggggctggctgtcattctgcacactgacagcaggaaggactcaccaccagctggaa

SEQ ID NO: 3 - DNA, 21 bp, Artificial sequence
  Synthetic oligonucleotide
acatgtcacttttgcttccct

SEQ ID NO: 4 - DNA, 23 bp, Artificial sequence
  Synthetic oligonucleotide
aggccatgctccatgcagactgc

SEQ ID NO: 5 - DNA, 20 bp, Artificial sequence
  Synthetic oligonucleotide
gggctgctttctggcatatg

SEQ ID NO: 6 - DNA, 20 bp, Artificial sequence
  Synthetic oligonucleotide
aggaaccacaggtgagggtg

SEQ ID NO: 7 - DNA, 20 bp, Artificial sequence
  Synthetic oligonucleotide
cctcacttctgtcacaggtc

SEQ ID NO: 8 - DNA, 23 bp, Artificial sequence
  Synthetic oligonucleotide
agaaggaaccacaggtgagggta

SEQ ID NO: 9 - DNA, 24 bp, Artificial sequence
  Synthetic oligonucleotide
agccccagagacccccaggcagtc

SEQ ID NO: 10 - DNA, 25 bp, Artificial sequence
  Synthetic oligonucleotide
ccacccgggagcccaagaaggtgcc

SEQ ID NO: 11 - DNA, 20 bp, Artificial sequence
  Synthetic oligonucleotide
gggagcccaagaaggtggcg

SEQ ID NO: 12 - DNA, 22 bp, Artificial sequence
  Synthetic oligonucleotide
ctggtgcttcaggttctcagtg

SEQ ID NO: 13 - DNA, 23 bp, Artificial sequence
  Synthetic oligonucleotide
cccgggagcccaagaaggtggca

SEQ ID NO: 14 - DNA, 35 bp, Artificial sequence
  Synthetic oligonucleotide
gtggtccgtactccacccaagtcgccgtcttccgc

SEQ ID NO: 15 - DNA, 19 bp, Artificial sequence
  Synthetic oligonucleotide
cgagtcctggcttcactcc

SEQ ID NO: 16 - DNA, 20 bp, Artificial sequence
  Synthetic oligonucleotide
ccatgccagacctgaagaac

SEQ ID NO: 17 - DNA, 20 bp, Artificial sequence
  Synthetic oligonucleotide
cttccaggcacagccatacc

SEQ ID NO: 18 - DNA, 23 bp, Artificial sequence
  Synthetic oligonucleotide
tgcccatgccagacctgaagaat

SEQ ID NO: 19 - DNA, 28 bp, Artificial sequence
  Synthetic oligonucleotide
gtcaagtccaagatcggctccactgaga

SEQ ID NO: 20 - DNA, 19 bp, Artificial sequence
  Synthetic oligonucleotide
cgagtcctggcttcactcc

SEQ ID NO: 21 - DNA, 20 bp, Artificial sequence
  Synthetic oligonucleotide
agaacctgaagcaccagcca

SEQ ID NO: 22 - DNA, 20 bp, Artificial sequence
  Synthetic oligonucleotide
cttccaggcacagccatacc

SEQ ID NO: 23 - DNA, 23 bp, Artificial sequence
  Synthetic oligonucleotide
ctgagaacctgaagcaccagccg

SEQ ID NO: 24 - DNA, 26 bp, Artificial sequence
  Synthetic oligonucleotide
ggaggcgggaaggtgagagtggctgg

SEQ ID NO: 25 - DNA, 20 bp, Artificial sequence
  Synthetic oligonucleotide
gctcattctctctcctcctc

SEQ ID NO: 26 - DNA, 20 bp, Artificial sequence
  Synthetic oligonucleotide
ggtgagggttgggacgggaa

SEQ ID NO: 27 - DNA, 25 bp, Artificial sequence
  Synthetic oligonucleotide
ccaggactcctccaccccatgcagc

SEQ ID NO: 28 - DNA, 23 bp, Artificial sequence
  Synthetic oligonucleotide
gaaggtgagggttgggacgggag

SEQ ID NO: 29 - DNA, 37 bp, Artificial sequence
  Synthetic oligonucleotide
ggtgcagggggtggaggagtcctggtgaggctggaac